(19) United States
(12) Patent Application Publication
(10) Pub. No.: US 2025/0219138 A1
(43) Pub. Date: Jul. 3, 2025

(54) ZWITTERIONIC ORGANIC FRAMEWORKS FOR SOLID-STATE SECONDARY BATTERY, ELECTROLYTE INCLUDING SAME, AND ALL-SOLID-STATE SECONDARY BATTERY INCLUDING SAME

(71) Applicant: INDUSTRY-UNIVERSITY COOPERATION FOUNDATION HANYANG UNIVERSITY ERICA CAMPUS, Ansan-si, Gyeonggi-do (KR)

(72) Inventors: Jong Ho KIM, Ansan-si, Gyeonggi-do (KR); Jun Hyeong LEE, Ansan-si, Gyeonggi-do (KR); Jae Hoon SHIN, Ansan-si, Gyeonggi-do (KR); Tae Woog KANG, Ansan-si, Gyeonggi-do (KR)

(21) Appl. No.: 18/843,222

(22) PCT Filed: Apr. 12, 2023
(86) PCT No.: PCT/KR2023/004934

§ 371 Date: Aug. 30, 2024

(30) Foreign Application Priority Data
Aug. 9, 2022 (KR) 10-2022-0098922

(51) Int. Cl. H01M 10/0565; H01M 10/052

(52) U.S. Cl. CPC H01M 10/0565; H01M 10/052; H01M 2300/0065; H01M 2300/0082

(57) ABSTRACT

Provided is a zwitterion organic framework for an all-solid-state secondary battery, and more specifically, a zwitterion organic framework for an all-solid-state secondary battery wherein the zwitterion organic framework is an organic framework, comprises a covalent bond and has a zwitterion structure.

### TECHNICAL FIELD

The present disclosure relates to a zwitterion organic framework for an all-solid-state secondary battery, an electrolyte comprising the same, and an all-solid-state secondary battery comprising the same, and more particularly, to a zwitterion organic framework for an all-solid-state secondary battery, which has excellent lithium-ion conductivity and stability even at room temperature, has a very simple synthesis process, and has a high synthesis yield, an electrolyte comprising the same, and an all-solid-state secondary battery comprising the same.

### BACKGROUND ART

Recently, solid electrolytes for secondary batteries are core technologies of all-solid-state secondary batteries that are receiving a lot of attention in the field of electric vehicles and future energy materials. As the electrolyte of such an all-solid-state secondary battery, an organic-based (dry polymer electrolyte), an inorganic-based (sulfide-based, etc.), a composite-based (nanoparticle filler and polymer), and the like have been studied. For example, Korean Patent Laid-Open Publication No. 10-2019-0033422 discloses a polymer electrolyte utilizing a polyethylene oxide (PEO)-based polymer or the like, but there are problems in that a manufacturing process is complicated according to the use of the polymer, it is difficult to effectively control desired ion characteristics, and stability is deteriorated.

However, the previously developed inorganic solid electrolyte has limitations in that it is very difficult to manufacture and process, contact resistance is very high due to poor contact with the surface of the electrode, and electrochemical stability is low. In order to overcome the disadvantages of the inorganic solid electrolyte, an organic solid electrolyte based on an amorphous polymer has been developed, but it is still difficult to commercialize it due to low ionic conductivity and thermal/electrochemical stability.

Therefore, it is necessary to develop a new solid electrolyte that has crystallinity without using a polymer based on a carbon material, exhibits very high ion conductivity at room temperature, and has very excellent thermal/electrochemical stability.

### DETAILED DESCRIPTION

### Technical Problem

An object of the present disclosure is to provide a new solid electrolyte for an all-solid-state secondary battery, which has crystallinity without using a polymer and has excellent ion conductivity and thermal stability even at room temperature, and a method of preparing the same.

However, the technical problem to be solved by the present disclosure is not limited to the above-mentioned problems, and other problems not mentioned will be clearly understood by those skilled in the art from the following description.

### Technical Solution

To achieve the above object, the present disclosure provides a zwitterion organic framework for an all-solid-state secondary battery, and more specifically, a zwitterion organic framework for an all-solid-state secondary battery wherein the organic framework is an organic framework, comprises a covalent bond and has a zwitterion structure.

In an embodiment of the present disclosure, the organic skeleton has crystallinity.

In an exemplary embodiment of the present disclosure, the organic skeleton has a zwitterion structure including a ring compound containing a cationic nitrogen as a ring element and an anionic functional group bonded to the ring compound.

In one embodiment of the present disclosure, the anionic functional group is linked to the cyclic compound by an alkyl group.

In an embodiment of the present disclosure, the zwitterion organic framework for an all-solid-state secondary battery for an all-solid-state secondary battery is any one of the following compounds.

The present disclosure provides an electrolyte comprising the above-described a zwitterion organic framework for an all-solid-state secondary battery.

The present disclosure also provides an all-solid-state secondary battery including the electrolyte described above.

### Advantageous Effects

The present disclosure provides a novel organic skeleton-based solid electrolyte having Zwitterion structure. The solid electrolyte according to the present disclosure has excellent lithium-ion conductivity and stability even at room temperature, and also has an advantage that a synthesis process is very convenient, and a synthesis yield is high. In addition, the all-solid lithium metal secondary battery comprising the solid electrolyte according to the present disclosure exhibits excellent cycling performance and high energy density.

### BRIEF DESCRIPTION OF DRAWINGS

FIG. 1 is a structural formula of an organic framework electrolyte having zwitterionic properties according to an embodiment of the present disclosure.

FIG. 2 is a result of structure analysis of a zwitterion organic framework according to an embodiment of the present disclosure and shows (a-c) TEM image of COF-A and (d-f) TEM image of Zwitt-COF-A1 of FIG. 1.

FIG. 3 is the results of pore and crystallinity analysis of a zwitterion organic framework (COF-B) according to an embodiment of the present disclosure and shows a) analysis of the size of Pore of COF-B and (b) the crystallinity of COF-B through XRD analysis.

FIG. 4 is a FT-IR spectrum for the Zwitt-COF-A1 of FIG. 1.

FIG. 5 shows the results of evaluating the ionic conductivity and high-temperature stability of a solid electrolyte including a zwitterion organic framework according to an embodiment of the present disclosure.

FIG. 6 illustrates the results of measuring the electrochemical stability of a solid electrolyte including a zwitterion organic framework according to an embodiment of the present disclosure.

FIG. 7 illustrates results of measuring lithium-ion adhesion and desorption performance according to various changes in current density of a lithium battery manufactured using a solid electrolyte including an amphoteric ion-organic skeleton according to an embodiment of the present disclosure.

FIG. 8 is a structure of (a) an all-solid lithium metal secondary battery manufactured using an amphoteric ion-covalent organic framework-based solid electrolyte, and (b) an example of the use of the manufactured all-solid-state lithium metal secondary battery.

FIG. 9 is a result of measuring cycling performance of an all-solid lithium metal secondary battery manufactured using a solid electrolyte including an amphoteric ion-organic framework according to an embodiment of the present disclosure.

### BEST MODE

Hereinafter, specific embodiments of the present disclosure will be described with reference to the drawings. However, this is only an example, and the present disclosure is not limited thereto.

In describing the present disclosure, when it is determined that a detailed description of a known technology related to the present disclosure may unnecessarily obscure the gist of the present disclosure, the detailed description thereof will be omitted. In addition, terms to be described below are terms defined in consideration of functions in the present disclosure and may vary depending on the intention or custom of a user or an operator. Therefore, the definition should be made based on the contents throughout this specification. In addition, the technical spirit of the present disclosure is determined by Claims, and the following embodiments are merely means for efficiently explaining the technical spirit of the present disclosure to those skilled in the art to which the present disclosure pertains.

The electrolyte according to the present disclosure imparts Zwitterion (zwitterion) to a Covalent organic framework (COF), thereby achieving excellent effects such as stability at room temperature and high ion conductivity.

The organic framework according to the present disclosure has a zwitterionic characteristic, and a zwitterionic ion is a neutral molecule having both electrically positive and negative electrical properties, and by forming both a cation and an anion in an organic framework having pores, the organic framework according to the present disclosure has a zwitterionic characteristic.

The organic framework structure according to an exemplary embodiment of the present disclosure is a cyclic compound having nitrogen as a cyclic element, and the nitrogen as a cyclic element is cationic, and another functional group (carboxyl group, sulfonyl group) connected to the organic framework by an alkyl group becomes anionic, and as a result, the organic framework according to the present disclosure has an amphoteric ion structure.

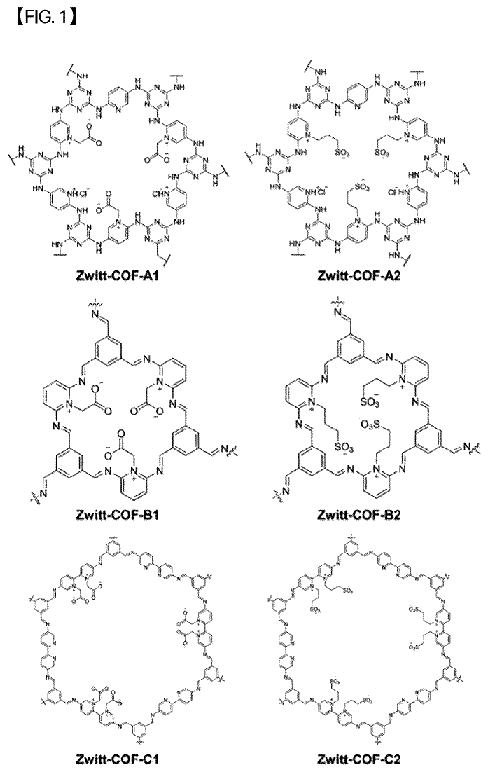

FIG. 1 is a structural formula of an organic framework electrolyte having zwitterionic properties according to an embodiment of the present disclosure.

Referring to FIG. 1, in the solid electrolyte according to the present disclosure, nitrogen of a cyclic compound constituting an organic framework has all anionic functional groups such as a cation, a carboxylate bonded to the organic framework, or a sulfonate, and the like, thereby having a zwitterion structure.

Since the present disclosure can be applied with various modifications and have various embodiments, specific embodiments will be illustrated in the drawings and described in detail in the detailed description. However, this is not intended to limit the present disclosure to specific embodiments, and the present disclosure covers all modifications, equivalents, and replacements included within the idea and technical scope of the present disclosure. In describing the present disclosure, when it is determined that a detailed description of a related known technology may obscure the gist of the present disclosure, the detailed description thereof will be omitted.

### Exemplary Embodiment

### COF-A Synthesis

2,5-diaminopyridine dihydrochloride was dissolved in CH3CN, and Diisopropylethylamine (DIPEA) was added thereto, and CAC (Cyanuric chloride) was added to 3-neck Rbf in an Ar atmosphere, and then CH3CN was added thereto.

Then, 2,5-diaminopyridine was slowly added to the CAC solution at ice bath. for 30 minutes, and then stirred at 25° C. for 1 hour. After raising the solution temperature to 85° C., the reaction was further progressed for 24 hours. After cooling down the reaction, the product was filtered and washed several times with acetone and n-hexane. Then, the product was dried in vacuum to synthesize an organic skeleton (COF-A) before having the amphoteric ion properties disclosed in FIG. 1. Here, A represents an organic framework of A1 and A2, and the following B and C represent an organic framework of B1/B2, C1, and C2, respectively. In addition, 1 and 2 are numbers for distinguishing compounds according to the type of amphoteric ions.

### COF-B, C Synthesis

1,3,5-triformylbenzene, 2,6-diaminopyridine or 5,5′-DIAMINO-2,2′-BIPYRIDINE was added to 3-neck Rbf and then dissolved in mesitylene/1,4-dioxane solution. After (2,6-diaminopyridine=used for the synthesis of COF-B, 5,5′-diamino-2,2′-bipyridine=used for the synthesis of COF-C), acetic acid was added to three-neck Rbf in an Ar atmosphere, and the temperature of the solution was raised to 120° C., and then the reaction was performed for 72 hours. After the reaction was cooled, the product obtained by the centrifuge was washed several times with tetrahydrofuran. The product was then dried in vacuo.

### Amphoteric Ionization of COF

The prepared COFs were dispersed in CH3CN containing Diisopropylethylamine (DIPEA), and the mixture was vigorously stirred and sonicated, COF-A was obtained by filtration, dispersed in CH3CN, and then sodium iodoacetate was added. Then, the resulting mixture was stirred at 40° C. for 3 hours, filtered to obtain the desired product, and the product was washed several times with water and then dispersed in acetone. Next, the product solution was centrifuged (10000 rpm, 30 minutes) to obtain a product, and then vacuum dried to synthesize COF-A1 of FIG. 1.

COF-A2 was synthesized in the same manner as in COF-A1, except that the indicated sodium iodoacetate was changed to 1,3-propene sultone.

For COF-B1 and COF-B2, a zwitterion structure was formed in the COF-B organic framework in the same manner as in COF-Al and COF-A2, and for COF-C1 and COF-C2, a zwitterion structure was formed in the COF-C organic framework in the same manner as in COF-A1 and COF-A2.

### Experimental Example

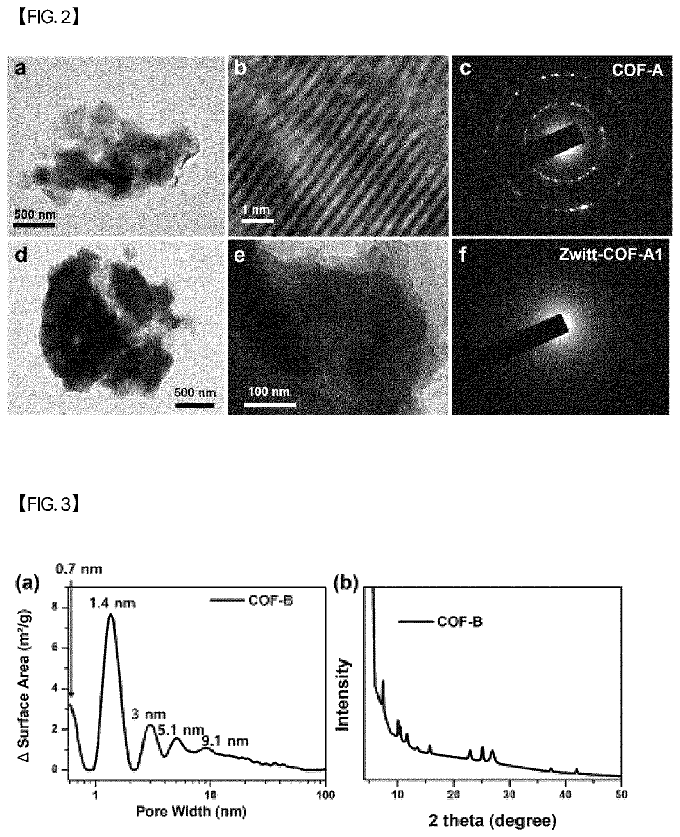

FIG. 2 is a TEM image of (a-c) an organic framework COF-A as a result of analyzing an organic framework structure of an electrolyte according to an embodiment of the present disclosure. (d-f) TEM images of Zwitt-COF-A1 having the zwitterionic structure of FIG. 1.

Referring to FIG. 2, it can be seen that the COF structure synthesized according to the present disclosure has crystallinity. In addition, the Zwitt-COF structure obtained after the zwitterion also has crystallinity and increased flexibility, thereby increasing the movement of lithium-ions, which will be described in more detail below.

FIG. 3 shows the results of pore and crystallinity analysis of an amphoteric ion-organic framework (COF-B) according to an embodiment of the present disclosure, (a) an analysis of the Pore size of COF-B. (b) A result of confirming crystallinity of COF-B through XRD analysis.

Referring to FIG. 3, the organic skeleton according to the present disclosure is a crystalline organic skeleton having pores.

Particularly, the present disclosure relates to an organic framework having an amphoteric ion-structure (Zwitt-COF) in which amphoteric ions are introduced, wherein the crystallinity of the organic framework is slightly reduced and the flexibility thereof is increased, and thus the organic framework is advantageous for lithium-ion conductivity, and the pore structure thereof is slightly changed but the organic framework of the present disclosure maintains a basic organic framework. In other words, when the chemical structure (unit cell structure & anion) of the zwitterion organic framework is changed, the crystallinity and pore size are also changed, and a specific channel is formed according to the stacking structure obtained by combining the organic frameworks, for example, the structure according to the combination of AA, AB, etc., to act as a movement path for Li ions. In addition, according to the chemical structure of the organic skeleton having an amphoteric ion structure, Li ion dissociation is smoother, thereby increasing lithium-ion conductivity.

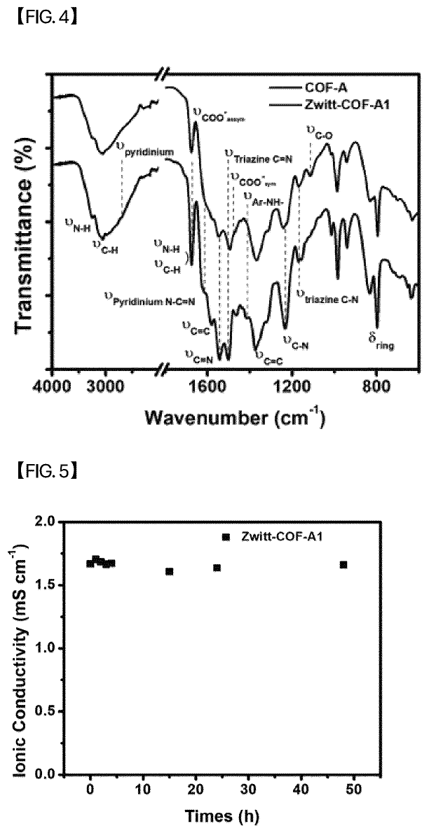

FIG. 4 is a FT-IR spectrum for the Zwitt-COF-A1 of FIG. 1.

Referring to FIG. 4, amphoteric ion properties were exhibited in the organic skeleton (COF) according to the reaction.

FIG. 5 shows the results of evaluating the ionic conductivity and high-temperature stability of a solid electrolyte including a zwitterion organic framework according to an embodiment of the present disclosure.

In more detail, 40% of a lithium-ion precursor (LiTFSI) based on the total mass is added to the Zwitt-COF-Al synthesized according to the above-described method, the resulting mixture is mixed well with a mortar, and then N-methyl pyrrolidone (NMP) mixed with polyvinylidene fluoride (PVDF), which is an aggregate, is added thereto to form a clay. This was put into a mold to make a circular electrolyte pellet and heated at 80 degrees Celsius to dry the solution and complete a solid electrolyte. FIG. 5 shows a result of measuring ionic conductivity at a high temperature (127 degrees Celsius, 400 K) using the solid electrolyte prepared as described above, and it can be confirmed that the solid electrolyte is stable by showing uniform conductivity at the corresponding temperature for 48 hours.

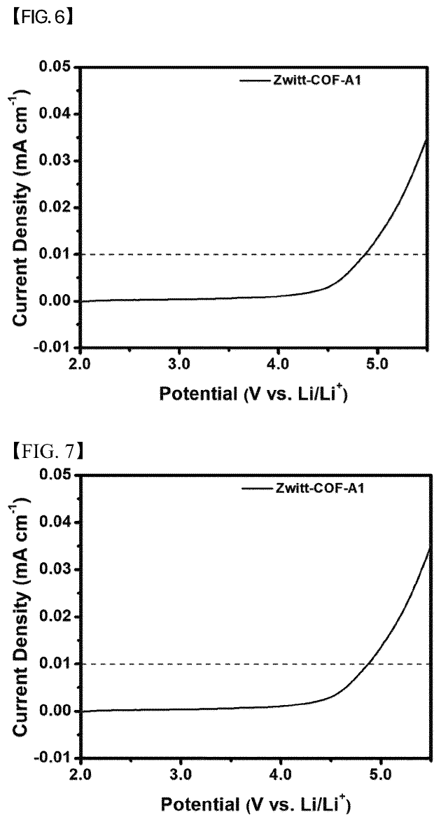

FIG. 6 illustrates the results of measuring the electrochemical stability of a solid electrolyte including a zwitterion organic framework according to an embodiment of the present disclosure. In order to be used as an electrolyte, only the role of moving lithium-ions when the electrolyte material is charged/discharged must be played, and oxidation/reduction must not occur in the material. Accordingly, in FIG. 6, the voltage range in which the oxidation reaction of the electrolyte does not occur was measured, and in FIG. 6, it was confirmed whether the oxidation reaction of the electrolyte occurs while changing the voltage when the lithium-ions are moved to the opposite side with the lithium metal on one side.

Referring to FIG. 6, it can be seen that the electrolyte according to the present disclosure is stable up to a voltage of 4.88 V. This means that a secondary battery having a high voltage of 4.88 V may be developed using the electrolyte, but this is merely an embodiment, and stability may be achieved even at a higher voltage through various changes in the amphoteric ions, and thus the secondary battery that may be manufactured in the present disclosure is not limited to 4.88 V.

FIG. 7 illustrates results of measuring lithium-ion adhesion and desorption performance according to various changes in current density of a lithium battery manufactured using a solid electrolyte including an amphoteric ion-organic skeleton according to an embodiment of the present disclosure.

In FIG. 7, in order to measure the adhesion and stripping performance of Li ions, a lithium electrode was disposed on both sides of the electrolyte according to the present disclosure, and a voltage applied while lithium-ions were repeatedly moved between both electrodes was measured.

Referring to FIG. 7, it can be seen that in the case of the red electrolyte having amphoteric ions according to the present disclosure, very stable voltage maintenance and low voltage are applied. That is, despite the same organic skeleton, it can be seen that it has very different characteristics depending on whether it has a zwitterion structure.

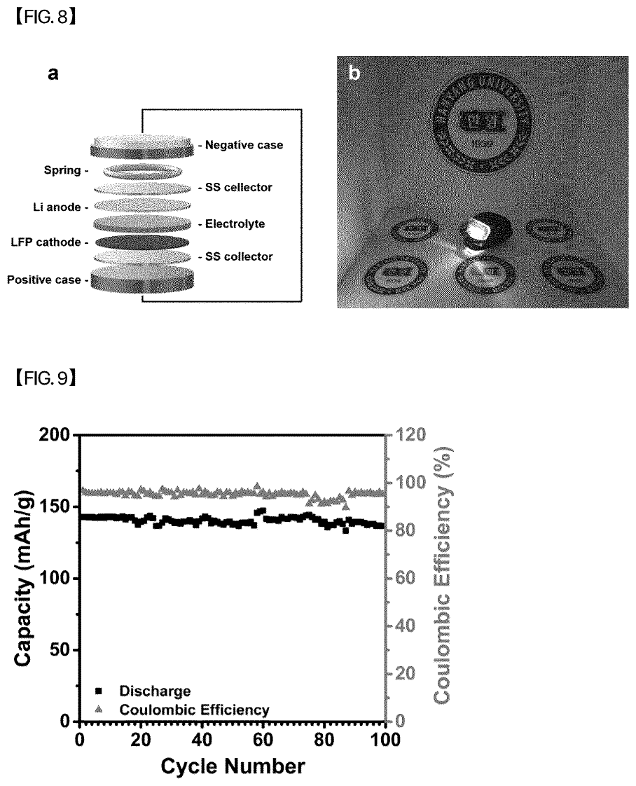

FIG. 8 is a structure of (a) an all-solid lithium metal secondary battery manufactured using a zwitterion covalent organic framework-based solid electrolyte. (b) An example of the use of the manufactured all-solid-state lithium metal secondary battery.

In FIG. 8, CR2032 was used as the case of the sub-battery, and a lithium metal was used as the anode and a LiFePO4 electrode was used as the cathode. An additional stainless steel (SS) collector and spring filled the empty space and served to assist the electrode and to press the structure to be well maintained, respectively.

As described above, the present disclosure provides a solid electrolyte having a zwitterionic structure, wherein the solid electrolyte binds TFSI- or BF4- in a lithium-ion precursor (LiTFSI, LiBF4, etc.), and lithium-ions are moved. According to the present disclosure, cations (N+) in the organic frameworks of Zwitt-COF, into which amphoteric ions are introduced, hold TFSI-, and anions in the organic frameworks electrostatically attract lithium-ions, thus allowing dissociation of the lithium-ion precursor to occur more effectively and easily. In addition, since an anion of an organic precursor is present in a channel formed by stacking of the zwitt-COF having both porosity and crystallinity, lithium-ions move well along the channel, and thus the zwitt-COF according to the present disclosure has high ion conductivity.

FIG. 9 is a result of measuring cycling performance of an all-solid lithium metal secondary battery manufactured using a solid electrolyte including an amphoteric ion-organic framework according to an embodiment of the present disclosure.

In FIG. 9, it was intended to confirm that the capacity and coulombic efficiency of the battery were maintained by measuring the cycling performance when the all-solid lithium metal secondary battery having the structure as shown in FIG. 8(a) was manufactured and charged and discharged at a speed of 0.2 C.

Referring to FIG. 9, the battery maintains a uniform capacity even when the electrolyte according to the present disclosure is charged and discharged 100 times at a speed of 0.2 C. In addition, it can be seen that even after the charging and discharging are performed 100 times, the coulombic efficiency indicating the ratio of the battery discharge capacity to the charge capacity is maintained close to 100%, so that the capacity reduction is significantly small. This proves that it is possible to manufacture a very stable lithium metal secondary battery using the electrolyte according to the present disclosure.

As a specific part of the present disclosure has been described in detail, it will be apparent to those skilled in the art that such a specific technique is merely a preferred embodiment, and the scope of the present disclosure is not limited thereby. Therefore, it will be said that the substantial scope of the present disclosure is defined by the appended claims and their equivalents.

1. A zwitterion organic framework for an all-solid-state secondary battery, wherein the zwitterion organic framework is formed by a covalent bond and has a zwitterion structure.

2. The zwitterion organic framework for an all-solid-state secondary battery according to claim 1, wherein the zwitterion organic framework has crystallinity for the all-solid-state secondary battery.

3. The zwitterion organic framework for an all-solid-state secondary battery according to claim 1, wherein the zwitterion organic framework has a zwitterion structure comprising a ring compound containing a cationic nitrogen as a ring element, and an anionic functional group bonded to the ring compound.

4. The zwitterion organic framework for an all-solid-state secondary battery according to claim 3, wherein the anionic functional group is bonded to a cyclic compound by an alkyl group.

5. The zwitterion organic framework for an all-solid-state secondary battery according to claim 1, wherein the zwitterion organic framework for the all-solid-state secondary battery is at least one of following compounds:

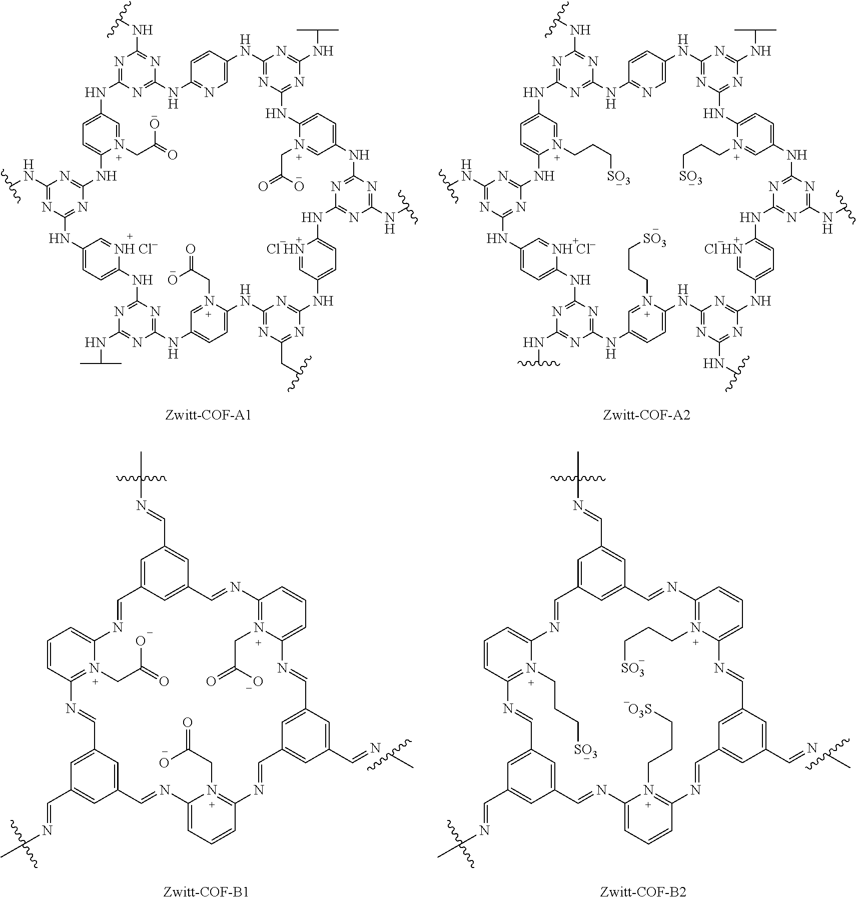

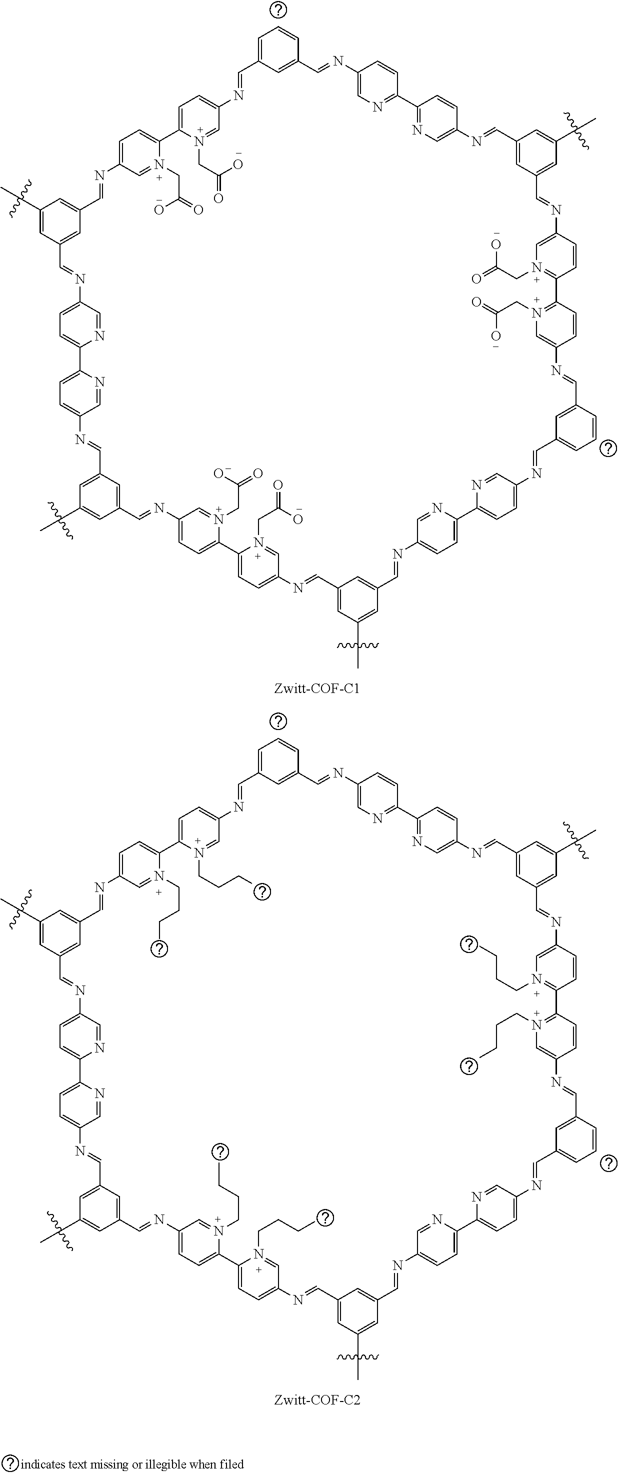

6. An electrolyte comprising the zwitterion organic framework for an all-solid-state secondary battery according to claim 1.

7. The electrolyte according to claim 6, wherein the zwitterionic organic framework in the electrolyte has a stacking structure.

8. The electrolyte according to claim 7, wherein a channel is formed in the stacking structure.

9. The electrolyte according to claim 8, wherein an organic precursor has an anion in the channel.

10. An all-solid-state secondary battery comprising the electrolyte according to claim 9.

11. The all-solid-state secondary battery according to claim 10, wherein the all-solid-state secondary battery is a lithium all-solid-state secondary battery, and lithium-ions of the lithium all-solid-state secondary battery move through the channel.

12. The all-solid-state secondary battery according to claim 11, wherein a moving speed of the lithium-ions is increased due to anions in the channel.

13. An electrolyte comprising the zwitterion organic framework for an all-solid-state secondary battery according to claim 5.

14. The electrolyte according to claim 13, wherein the zwitterionic organic framework in the electrolyte has a stacked structure.

15. The electrolyte according to claim 14, wherein a channel is formed in the stacked structure.

16. The electrolyte according to claim 15, wherein the channel has an anion of an organic precursor.

17. An all-solid-state secondary battery comprising the electrolyte according to claim 16.

18. The all-solid-state secondary battery of claim 17, wherein the all-solid-state secondary battery is a lithium all-solid-state secondary battery, and lithium-ions of the lithium all-solid-state secondary battery move through the channel.

19. The all-solid-state secondary battery of claim 18, wherein a moving speed of the lithium-ions increases due to anions in the channel.
